(19) United States
(12) Patent Application Publication
(10) Pub. No.: US 2006/0020199 A1
(43) Pub. Date: Jan. 26, 2006

(54) Urethral radiation treatment system and method

(75) Inventors: James B. Stubbs, Alpharetta, GA (US); Bartolome J. Salazar, Alpharetta, GA (US); Gregory K. Edmundson, Rough And Ready, CA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: CYTYC CORPORATION, Marlborough, MA (US)

(21) Appl. No.: 11/152,824

(22) Filed: Jun. 14, 2005

Related U.S. Application Data
(60) Provisional application No. 60/579,357, filed on Jun. 14, 2004.

(51) Int. Cl. A61B 5/05

(52) U.S. Cl. 600/411

(57) ABSTRACT

Provided herein is an applicator system and method for treating tissue via a body lumen. The applicator can include a graduated member having an anchor configured to position the graduated member in a predetermined relationship relative to an anatomical feature and at least one visualizable marker. The system can also include a visualization tool for locating a target tissue site relative to the at least one visualizable marker, and a treatment catheter having an anchor configured to correspond to the graduated member anchor.

### CROSS-REFERENCE TO RELATED APPLICATIONS

The present invention claims priority to U.S. Provisional Application Ser. No. 60/579,357, filed on Jun. 14, 2004 and entitled “Urethral Radiation Treatment Systems and Method,” which is hereby incorporated by reference in its entirety.

### FIELD OF THE INVENTION

The invention relates generally to methods and applicators for use in treating a patient's urethra, and more particularly to an apparatus for the location and treatment of or from within the urethra by the application of radiation.

### BACKGROUND OF THE INVENTION

A number of situations arise in which it may be desirable to treat (or to treat other tissue from within) a patient's urethra/ureter with therapeutic rays. One example is the treatment of the prostate. The prostate is a solid organ which surrounds the urethra of the male human between the base of the bladder and the urogenital diaphragm. Benign prostatic hypertrophy (BPH) is a common condition among male humans aged 45 or older. Prostate cancer is a leading cause of death among males, and can frequently be diagnosed with the aid of a simple blood antigen-detecting test. Radiation therapy and prostatectomy are the primary treatments available for prostate cancer and prostatectomy is currently the primary treatment for BPH. Prostatectomy has numerous drawbacks, which have been widely described in the art. External beam irradiation of the prostate for the treatment of localized prostate cancer is associated with small bowel injury, radiation proctitis, and urethral stricture (Gibbons et al., 1979, J. Urol. 121:310-312). At least two groups have employed transurethral radiation therapy as a supplement to external beam irradiation of localized prostate cancer tissue (Harada et al., 1993, Rad. Oncol. 11:139-145; Skarlatos et al., 1994, Urol. Int. 53:209-213). In addition, another group has employed transurethral radiation therapy as a sole treatment for recalcitrant BPH-related urine retention (Koukourakis et al., 1994, Med. Dosimetry 19:67-72). Each of these groups employed ultrasonography, computerized tomography, or fluoroscopy imaging methods to identify the tissue to be treated or to confirm the position of the radiation source relative to the tissue to be treated. Identification of the location of tissue in need of treatment and placement of a radiation source using one of these imaging methods is dependent upon the deformability of the tissues being imaged, the body posture of the subject during the identification or placement, the position of the imaging device, and other factors which may not be easily replicated.

Failure to precisely control the amount and location of transurethrally-delivered radiation can result in damage to the urethra itself or to other organs located in close proximity thereto, including the bladder, rectum and the prostate. It is thus critical to identify the position of a tissue in need of treatment and the location of radiation source as accurately as possible.

Another example of the delivery of therapeutic rays through the urethra/ureter is the treatment of urethral and ureteric stricture. Urethral stricture is a common complication of urological procedures, particularly following urethral intervention by an urologist (Baskin et al., 1993, J. Urology 150:642-647; Stormont et al., 1993, J. Urology 150:1725-1728). Formation of a urethral stricture is thought to involve disruption of the urothelium, followed by hypertrophy of urothelial or other tissues, resulting in stenosis. A urethral stricture may also be formed by hypertrophy of a tissue located in close proximity to the urothelium, such as prostate tissue or corpus spongiosum penis tissue in male humans or muscle tissue or spongiose erectile tissue in female humans. Non-limiting examples of urological interventions known to be associated with urethral stricture include transurethral resection of the prostate, radical prostatectomy, external beam irradiation of prostate tissue, and other urological interventions which disturb the urethra. Non-limiting examples of diseases or disorders known to be associated with urethral stricture include BPH, prostate cancer, internal/external trauma, certain infections and urethral cancer. Further details of tissues which comprise the urethra or which are located in close proximity thereto in the human are found in, for example, Williams et al., eds. (1980, Gray's Anatomy, 36th ed., W.B. Saunders Co., Philadelphia, pp. 1408-1409).

Known treatments for urethral strictures include surgical modification of the urethra, laser-assisted modification of the urethra, urethroplasty, and urethral stent implantation (Bosnjakovic et al., 1994, Cardiovasc. Intervent. Radiol. 17:280-284; Badlani et al., 1995, Urology 45:846-856; Mundy, 1989, Brit. J. Urology 64:626-628; Quartey, 1993, Ann. Urol. 27:228-232).

Ureteric stricture is another known complication of urological procedures and of disease and disorder states. Ureteric strictures may involve hyperplasia or hypertrophy of any of the tissue layers of a ureter, namely the fibrous layer, the muscular layer, or the mucous layer, or may involve hyperplasia or hypertrophy of a tissue or organ located in close proximity to a ureter. Further details of tissues which comprise a ureter or which are located in close proximity thereto in the human are found in, for example, Williams et al., eds. (1980, Gray's Anatomy, 36th ed., W.B. Saunders Co., Philadelphia, pp. 1402-1404). Surgical treatments are known for treatment of ureteric stricture.

Bladder neck contracture (BNC) is another condition that can be treated through the urethra and/or bladder. BNC can arise as a complication of urological procedures in which scar tissue forms near the bladder neck and blocks or inhibits the passage of fluid from the bladder. Treatments can include removal of the scar tissue and/or resection of the tissue around the scar tissue.

U.S. Pat. No. 6,607,477 to Longton et al. provides methods and systems for placing a source of therapeutic rays in the urethra at a desired location. In particular, a kit comprising a matched pair of graduated catheters is disclosed, wherein a graduated locator catheter can provide a known relationship to a position within a bodily lumen. The present invention further elaborates on this method and provides further systems for use in the method described above and herein.

### SUMMARY

Described herein are applicator systems and methods for treating tissue via a body lumen. In one embodiment, an applicator system includes a graduated member configured for placement in contact with an anatomical feature, the graduated member including markers and an anchor. Positioning the graduated member relative to an anatomical feature allows a user to determine the location of a target tissue site relative to the anatomical feature. The system can further include a visualization tool for locating the target tissue site relative to the markers.

Once the location of the target tissue site, relative to the anatomical feature, is known, a treatment catheter can be positioned to deliver a therapeutic dose of radiation. In one aspect, the treatment catheter includes an anchor that can mate with the same anatomical feature as the anchor of the graduated member. With the catheter anchor positioned against the anatomical feature, the location of the target tissue site can be determined relative to the catheter.

In one aspect, the graduated member includes a series of markers that can be visualized with a surgical scope. For example, the markers can be a series of color-coded bands and the visualization tool can be a surgical scope configured to visualize the interior of a body lumen. In another aspect, the graduated member can be disposed alongside the surgical scope. Alternatively, the graduated member can be adapted to pass through a lumen of the surgical scope.

The anchor positioned on the graduated member can be adapted to move between an expanded and a contracted position. In the contracted position, the anchor can pass through the lumen of a surgical scope and into a body lumen or cavity. Once positioned within the body cavity, the anchor can move into an expanded position and can mate with an anatomical feature.

In one embodiment, the system further comprises a second graduated member disposed within or adjacent to the treatment catheter and a second visualization tool for confirming the position of the treatment catheter by visualizing the second graduated member. For example, the second graduated member can be compatible with an imaging technique and the second visualization tool can be an imaging device. In one aspect, the second graduated member is visualized using x-rays.

Further described herein is an applicator system comprising a graduated member having an anchor configured to establish a predetermined relationship between the graduated member and an anatomical feature. In one aspect, the graduated member can include one or more visualizable markings. The system can further include a visualization tool for locating a treatment site relative to the graduated member, a treatment catheter, and a second graduated member. The second graduated member can be disposed within or adjacent to the treatment catheter and can be visualized via a second visualization tool.

A method for treating tissue through a body lumen is also described herein. The method can include the steps of providing a graduated member having an anchor and visualizable markings and positioning the anchor against an anatomical feature. With the graduated member in place, a user can map the location of a target tissue site relative to the visualizable markings. The mapping procedure allows a user to determine the position of the target tissue with respect to the anatomical feature.

In one aspect, the method further includes positioning a treatment catheter relative to the anatomical features and delivering therapeutic radiation. For example, an anchor on the treatment catheter can be positioned relative to the anatomical feature to hold the treatment catheter in place. Based on the location of the target tissue relative to the anatomical feature, determined during the mapping procedure, a user can accurately and precisely deliver a dose of therapeutic radiation from the catheter to the target tissue site.

### BRIEF DESCRIPTION OF THE DRAWINGS

The invention will be more fully understood from the following detailed description taken in conjunction with the accompanying drawings, in which:

FIG. 1A is a side view of one embodiment of the graduated member disclosed herein;

FIG. 1B is a side view of the graduated member of FIG. 1A showing an anchor in an expanded position;

FIG. 2 is a cut-away view of a bladder and urethra;

FIG. 3 is a cut-away view of the bladder and urethra of FIG. 2 with a visualization tool positioned therein;

FIG. 4 is another illustration of the bladder and urethra of FIG. 3 with the distal end of the visualization tool positioned in the bladder;

FIG. 5 illustrates a graduated member being delivered to the bladder through a visualization tool;

FIG. 6 illustrates the graduated member of FIG. 5 with the anchor in an expanded position;

FIG. 7 illustrates the graduated member of FIG. 6 with the anchor positioned against the wall of the bladder;

FIG. 8 illustrates the graduated member in position for determining the location of a target tissue site;

FIG. 9 is a side view of the graduated member positioned within the urethra and in position for determining the location of the target tissue site;

FIG. 10 is a side view of the anchor of the graduated member moving from an expanded position to a contracted position;

FIG. 11 is a side view of a catheter being positioned with the urethra;

FIG. 12 is a side view of the catheter of FIG. 11 with an anchor in the expanded position;

FIG. 13 is a side view of the catheter of FIG. 12 with a second anchor in the expanded position;

FIG. 14 is a side view of the catheter with a second graduated member positioned therein; and

FIG. 15 is a side view of the catheter with a therapeutic radiation source positioned therein.

### DETAILED DESCRIPTION OF THE INVENTION

Disclosed herein are systems and methods for mapping a target tissue site within a body lumen. In one embodiment, a tool capable of visualizing the target tissue and measuring the distance between the target tissue and an anatomical feature is used to map the target tissue. The tool can include a graduated member comprising an anchor and visualizable markings. Positioning the graduated member in a fixed position relative to an anatomical feature allows a user to determine the relative location of the target site by referencing the visualizable markings. This information facilitates later placement of a radiation delivery device, such that, a therapeutic dose of radiation can be precisely and accurately delivered to the target tissue. The improved positioning of the radiation source, made possible by the graduated member, can reduce the exposure of healthy tissue and assure that the target tissue receives the desired dose of radiation.

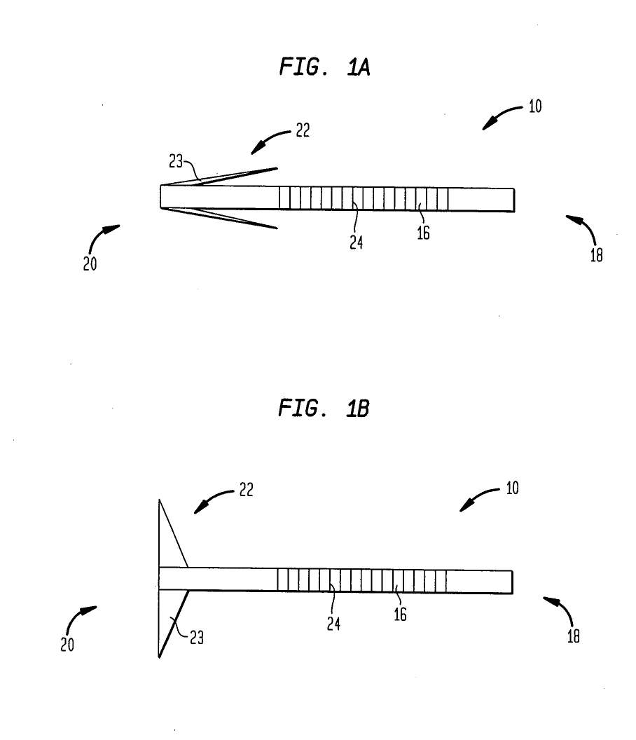

FIGS. 1A and 1B illustrate one embodiment of a graduated member 10 including an elongate body 16 with a proximal end 18 and a distal end 20. Distal end 20 can be adapted to mate with an anatomical feature(s) and can include a mating element. For example, distal end 20 can include an anchor 22 that is sized and shaped to contact an internal body feature.

In one aspect, anchor 22 can move between a contracted position (FIG. 1A) and an expanded position (FIG. 1B). In the expanded position, anchor 22 is sized for positioning against an anatomical feature, and in the contracted position anchor 22 is sized for movement through a body lumen or another device. For example, anchor 22 can include arms 23 as shown in FIGS. 1A and 1B. In the contracted position, anchor arms 23 can be positioned against elongate body 16 such that anchor 22 has a minimal profile to facilitate insertion into a body lumen. In the expanded position, arms 23 can open and be adapted for engaging an anatomical feature.

Movement of anchor 22 between the expanded and contracted positions can be achieved with a variety of mechanisms. In one aspect, arms 23 are biased in the expanded position, such that when unhindered, arms 23 will move into the expanded position. For example, arms 23 can be formed from a resilient material that will spring into the expanded position when not held in the contracted position. Alternatively, arms 23 could be formed of a shape memory material. When activated, the shape memory material can move arms 23 to the expanded position. For example, arms 23 could be formed from a heat activated shape memory material such that when the temperature of the shape memory material rises above an activation temperature, the arms will move into the expanded position. In one exemplary embodiment, the activation temperature can be below body temperature such that body heat activates arms 23. In another embodiment, arms 23 could be mechanically actuated.

While the mating element of graduated member 10 is illustrated as an anchor, one skilled in the art will appreciate that a variety of other mechanisms could be used to engage anatomical features. For example, the mating element could alternatively, or additionally, include a balloon, umbrella-shaped mechanism, scaffold or other distensible member. In one embodiment, anchor 22 is a balloon.

Elongate body 16 is preferably sized and shaped for insertion into a body lumen. In one exemplary embodiment, body 16 is defined by a rigid wire that extends between anchor 22 and proximal end 18.

Elongate body 16 can further include indicia useful for determining a position on the body 16 relative to anchor 22. For example, FIGS. 1A and 1B illustrate a series of markers 24 positioned along body 16. In use, markers 24 allow a user to measure the position of target tissue relative to an anatomical feature. In one embodiment, markers 24 can include a series of coded bands (i.e., color and/or shape coded bands), which allow for visual determination of the location of target tissue. Alternatively, or additionally, markers 24 can include ridges and/or depressions which can provide tactile feedback to the user.

Proximal end 18 of graduated member 10 can be adapted for grasping by a user and/or for mating with another device. In one embodiment, proximal end 18 can include a handle (not illustrated) that can facilitate grasping by a user such that a user can control the movement of graduated member 10 through a body lumen.

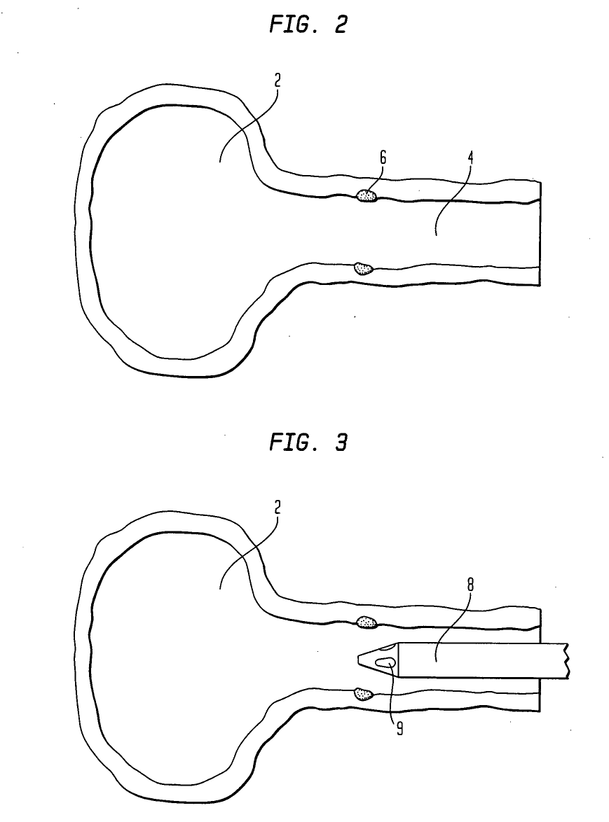

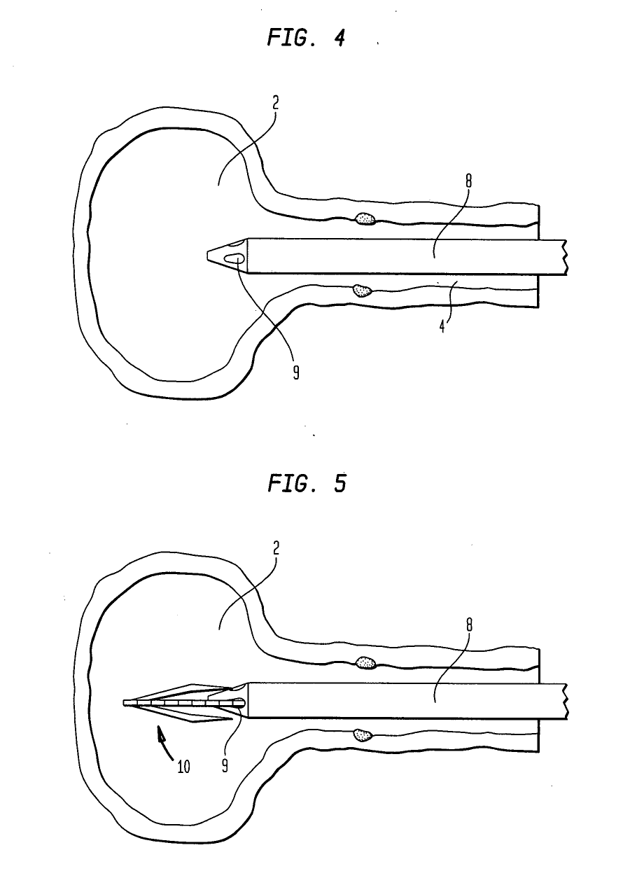

In one embodiment, graduated member 10 is used to map the position of target tissue relative to an anatomical feature as illustrated herein by reference to FIGS. 2 through 16. FIG. 2 illustrates a patient's bladder 2 (to the left) and urethra 4 (extending to the right (the FIGS. are not to scale)), with a lesion 6 illustrated on the inner wall of the patient's urethra. In FIG. 3, a visualization tool 8 has been deployed to resect the lesion in the wall of the patient's urethra. In one embodiment, visualization tool 8 is a cystoscope that includes multiple tools, such as, a camera, a light source, and at least one tool lumen 9. For the purpose of resecting the lesion, a cutting or scraping tool can be deployed through tool lumen 9 or alongside visualization tool 8 so that the surgeon can see the region being resected. After resection, the visualization tool can then be advanced into the bladder as illustrated in FIG. 4.

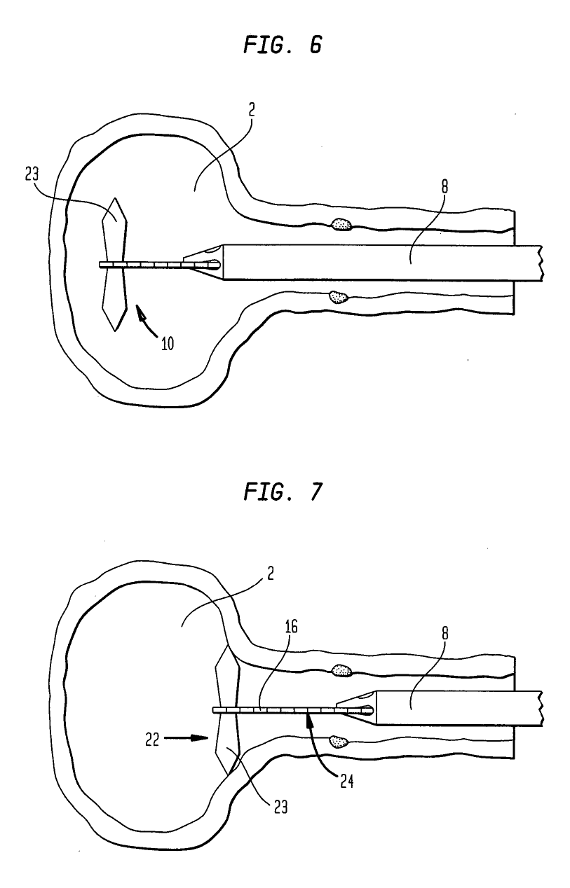

As shown in FIG. 5, graduated member 10 can be deployed through lumen 9 in visualization tool 8. The small profile of anchor 22 in the closed (contracted) position allows it to pass through lumen 9 and into the bladder. After anchor 22 moves out of lumen 9, it can expand as shown in FIG. 6. In one embodiment, arms 23 of anchor 22 are resilient such that after anchor 22 moves out of the confines of lumen 9, arms 23 spring into position. Alternatively, arms 23 could be spring loaded or mechanically controlled. In yet another embodiment, arms 23 are formed of a shape memory material. After exiting from lumen 9, arms 23 expand into a position for engaging an anatomical feature. One skilled in the art will appreciate that arms 23 of anchor 22 can be moved into an expanded position with a variety of mechanisms.

Once deployed, anchor 22 is preferably configured to lodge against the bladder wall at the opening of the urethra. One skilled in the art will appreciate that anchor 22 can be seated against a variety of anatomical features, such as, for example, the bladder wall, the bladder neck, the urethra, the ureter, and combinations thereof. Visualization tool 8 and graduated tool 10 are then drawn back through the urethra so that anchor 22 sits against the bladder wall as illustrated in FIG. 7.

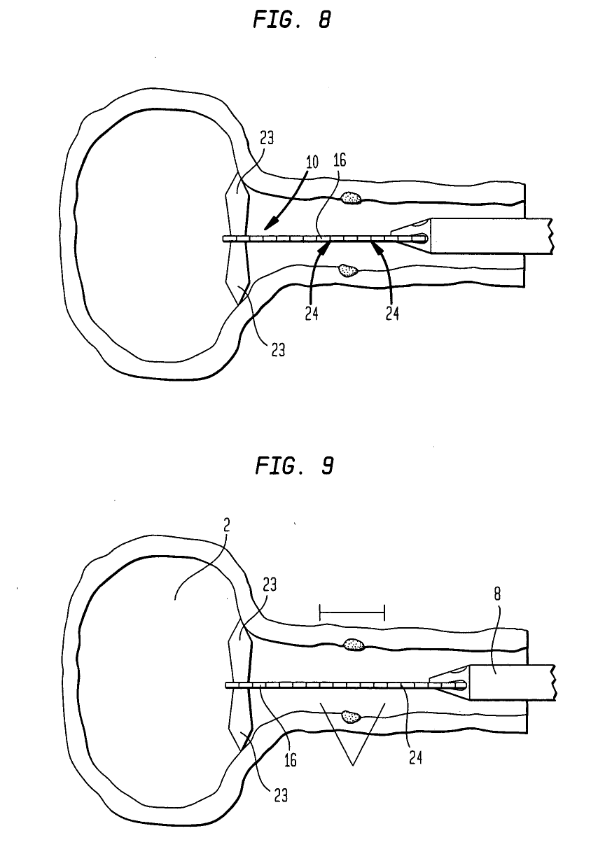

The surgeon can next use visualization tool 8 to compare the location of the resection to makers 24 on graduated member 10 in order to locate the position of the resection with respect to anchor's 22 location. One skilled in the art will appreciate that visualization tool 8 can be anything that allows direct or indirect visualization of the urethra, like a camera or the human eye viewing through the a cystoscope's optics. The visualization tool can be the same as, or separate from, the tool through which graduated member 10 is delivered. As illustrated in FIGS. 8 and 9, the visualization tool allows a user to find the location of the resection (i.e., target tissue) relative to markers 24.

Where the visualization tool is a camera that is internal to the urethra, one useful form of markings is color-coded bands positioned at known locations along elongate body 16. In this way, the surgeon can visualize the resection, then the colored bands in order to map the resection location to a known position within the urethra based on predetermined spatial relationship between the colored bands and the anchor. The markings can also be in the form of letters or symbols that can optionally have different colors to differentiate separate locations along the graduated tool. In another embodiment, markers 24 are ridges and/or recesses which provide tactile feedback. For example, visualization tool 8 can be dragged against ridges and/or recesses on graduated member 10 to provide tactile feedback through the visualization tool.

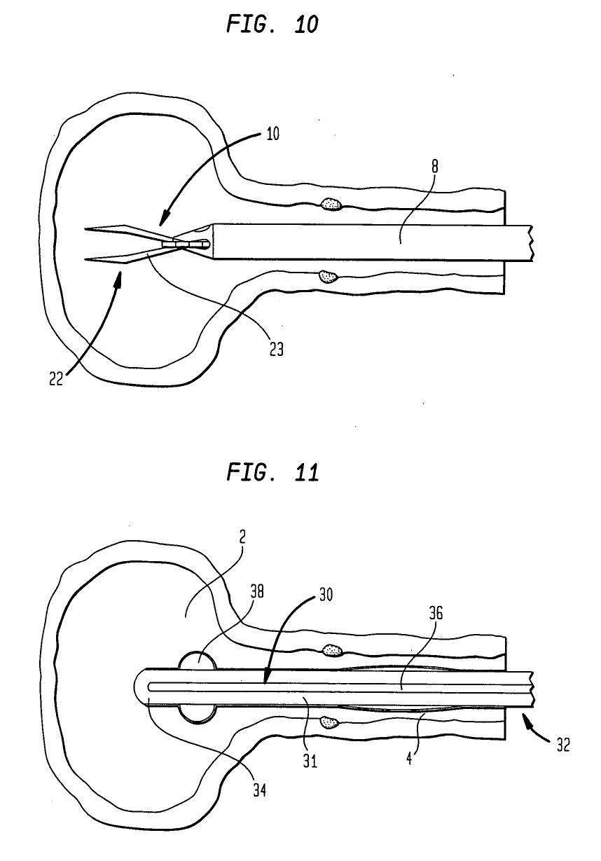

After the location of the target tissue site has been determined (i.e., mapped), the graduated member and the visualization tool can be removed. As illustrated in FIG. 10, anchor 22 can be closed by advancing visualization tool 8 over graduated member 10 causing anchor 22 to fold closed. For example, arms 23 can be biased in the open position and resist closing such that the force of anchor 22 against the bladder wall will not cause the arms to fold. When a user wishes to close anchor 22, visualization tool 8 can be used to apply sufficient force to move arms 23 into a contracted position as shown in FIG. 10

With the target tissue mapped via graduated member 10, a radiation therapy device can be deployed. The information gathered from markings 24 on graduated member 10 can help a surgeon to properly position the radiation therapy device.

In one embodiment, the radiation therapy device is a catheter 30 as illustrated in FIG. 11. Catheter 30 includes a generally elongate body 31 having a proximal end 32, a closed distal end 34, and an internal lumen 36 for receiving a radiation source. Distal end 34 of catheter 30 can include mating features adapted to engage an anatomical feature. In one embodiment, catheter 30 includes a mating feature configured to mate with the same anatomical feature as anchor 22. For example, catheter 30 can include a balloon 38 adapted to mate with a bladder wall. When balloon 38 is mated with the bladder wall, the spacing between the bladder wall and the target tissue can be determined based on the mapping procedure performed with graduated member 10.

The mating feature of catheter 30 can be the same or different from anchor 22 of graduated member 10. For example graduated member 10 and catheter 30 can both include an anchor or balloon.

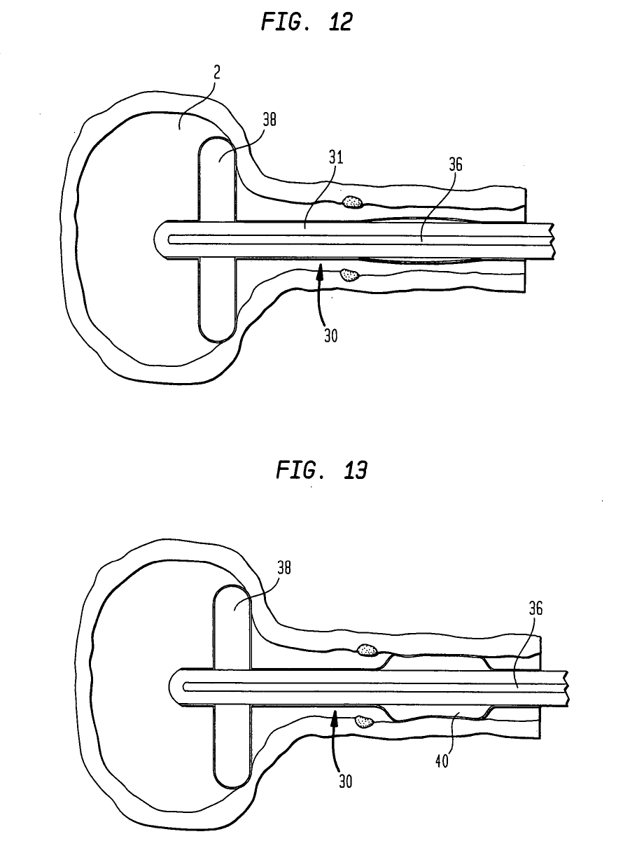

As shown in FIG. 12, catheter 30 can be positioned with in the urethra such that distal end 34 of catheter 30 is positioned within the bladder and balloon 38 engages the bladder wall. As shown in FIG. 13, an optional proximal anchor balloon 40 may also be expanded in order to prevent catheter 30 from sliding deeper into the bladder. Proximal balloon 40 can also be used under some circumstances to urge tissue that will be treated into or toward a desired formation.

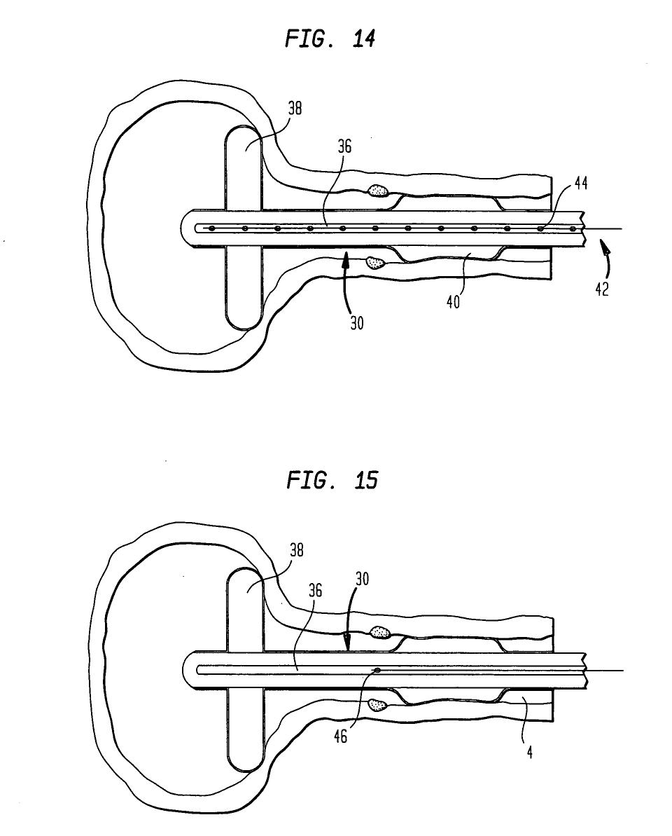

In one embodiment, the location of the target tissue relative to the anatomical feature (i.e., the bladder wall), determined from graduated member 10, allows a user to find the distance between the proximal end of catheter 30 and the location of the target tissue. Based on this information, a radiation source can be positioned within internal lumen 36. However, it may also be desirable to provide a second graduated tool 42 and a second visualization tool to establish offset and dwell positions for the source of therapeutic rays. As illustrated in FIG. 14, a “dummy” seed train 42 can be employed for this purpose. The dummy seed train has markings 44 that can be viewed using external visualization (x-ray or fluoroscopic visualization for example) to confirm the position of the treatment catheter. Finally, one or more sources of therapeutic rays 46 can be deployed as illustrated in FIG. 15 and described in the aforementioned U.S. Pat. No. 6,607,477. One skilled in the art will appreciate that the source of therapeutic radiation can include, for example, gamma/x-ray sources, beta sources, manmade ionizing/nonionizing radiation, and combinations thereof.

A person of ordinary skill in the art will appreciate further features and advantages of the invention based on the above-described embodiments. For example, specific features from any of the embodiments described above, as well as feature disclosed in the above referenced U.S. Pat. No. 6,607,477, may be incorporated into systems or methods of the invention in a variety of combinations and subcombinations, as well as features referred to in the claims below which may be implemented by means described herein. In particular, the graduation and visualization means may be used in any number of combinations, as well as the sources of therapeutic rays, from any of these sources. Accordingly, the invention is not to be limited by what has been particularly shown and described, except as indicated by the appended claims or those ultimately provided. Any publications and references cited herein are expressly incorporated herein by reference in their entirety.

1. An applicator system for treating tissue via a body lumen, comprising:
a graduated member having an anchor configured to position the graduated member in a predetermined relationship relative to an anatomical feature, the graduated member further having at least one visualizable marking;
a visualization tool for locating a target tissue site relative to the graduated member; and
a treatment catheter having an anchor configured to correspond to the graduated member anchor.

2. The system of claim 1, further comprising a source of therapeutic rays disposed in the treatment catheter.

3. The system of claim 1, further comprising a second graduated member disposable within or along with the treatment catheter and a second visualization tool for confirming the position of the treatment catheter by visualizing the second graduated member.

4. The system of claim 1, wherein the visualization tool is a surgical scope configured to visualize the interior of the body lumen.

5. The system of claim 4, wherein the graduated member is disposable alongside the surgical scope.

6. The system of claim 4, wherein the graduated member is adapted to pass through a lumen of the surgical scope for deployment within a body lumen.

7. The system of claim 1, wherein the anchor is adapted to move between an expanded position and a contracted position.

8. The system of claim 7, wherein the anchor is biased in the expanded position.

9. The system of claim 1, wherein the at least one visualizable marking is a series of color-coded bands.

10. An applicator system, comprising:
a graduated member having an anchor configured to establish a predetermined relationship between the graduated member and an anatomical feature, the graduated member further having one or more visualizable markings;
a visualization tool for locating a treatment site relative to the graduated member;
a treatment catheter; and
a second graduated member disposable within with the treatment catheter and a second visualization tool for confirming the position of the treatment catheter by visualizing the second graduated member.

11. A method for treating tissue through a body lumen, comprising:
providing a graduated member having an anchor and visualizable markings;
positioning the anchor against an anatomical feature; and
mapping the location of a target tissue site relative to the visualizable markings to determine the relative position of the target tissue with respect to the anatomical feature.

12. The method of claim 11, further comprising the step of moving the graduated member through a lumen in a visualization device and into a body cavity.

13. The method of claim 12, further comprising the step of opening the anchor within the body cavity.

14. The method of claim 11, further comprising the step removing the graduated member and positioning a treatment catheter.

15. The method of claim 14, wherein a source of therapeutic radiation is positioned within the treatment catheter.

16. The method of claim 15, wherein the source of therapeutic radiation is selected from the group consisting of gamma/x-ray sources, bata sources, manmade ionizing radiation, and combinations thereof.
